(19) United States
(12) Patent Application Publication
(10) Pub. No.: US 2009/0112300 A1
(43) Pub. Date: Apr. 30, 2009

(54) REDUCED BENDING STIFFNESS POLYURETHANE TUBING

(76) Inventor: Mitchell L. Horn-Wyffels, New Hope, MN (US)

Correspondence Address: FAEGRE & BENSON, LLP;ATTN: PATENT DOCKETING (32469), 2200 WELLS FARGO CENTER, 90 SOUTH SEVENTH STREET, MINNEAPOLIS, MN, 55402-3901, US

(21) Appl. No.: 12/195,019

(22) Filed: Aug. 20, 2008

Related U.S. Application Data
(60) Provisional application No. 60/983,434, filed on Oct. 29, 2007.

(51) Int. Cl. A61B 18/14

(52) U.S. Cl. 607/116

(57) ABSTRACT

A medical electrical lead body having an outer diameter of less than 5 French includes a reinforcing member. The reinforcing member maintains the axial load bearing capability of the lead body while at the same time providing for flexibility of the lead body.

### TECHNICAL FIELD

The present invention relates to medical electrical leads. More particularly, the present invention relates to the construction of medical electrical lead bodies having reduced outer diameters.

### BACKGROUND

Implantable medical devices for treating irregular contractions of the heart with electrical stimuli are well known. Exemplary implantable devices are defibrillators and pacemakers. Various types of electrical leads for defibrillators and pacemakers have been suggested. Such leads have an elongated, flexible body and are introduced into the patient's vasculature at a venous access site and travel through veins to the sites where the leads' electrodes will be implanted or otherwise contact target coronary tissue. Therapy can be delivered to either the right side or the left side of the heart.

Recently, there has been an effort to reduce the outer diameter of medical electrical lead bodies including endocardial, cardioversion, and defilibration leads. Reduction in lead body diameter can facilitate placement of the lead at a target location within a patient's body. However, a reduction in lead body diameter may compromise the axial load bearing capability and the tear resistance of the lead. When constructing lead bodies having diameters of about 5 French or less, it is desirable to maintain sufficient flexibility for maneuverability through a patent's venous system while at the same time providing a lead body having a high tensile strength and tear resistance.

### SUMMARY

According to one embodiment, the present invention is a medical electrical lead including a lead body, a conductor, and a reinforcing member. The lead body includes a proximal end configured to be connected to a pulse generator and a distal end. The conductor extends from the proximal end to the distal end of the lead body. At least one electrode is operatively connected to the conductor. The reinforcing member is disposed over a least a portion of the conductor, and includes a tubular member having at least one recess formed in an outer surface thereof.

According to another embodiment, the present invention is a medical electrical lead including a reinforcing member disposed over the first insulator and extending from substantially the proximal end to the distal end of the lead body. The reinforcing member includes a tubular member defining a lumen and including an outer surface having a non-random, ordered pattern of a plurality of recesses extending through the outer surface into the lumen.

According to yet another embodiment, the present invention is a medical electrical lead including at least one flexibility region located along the lead body. The flexibility region includes at least one recess formed in an outer surface of a tubular member disposed over at least a portion of the first insulator. The flexibility region may vary in flexibility from the proximal end to the distal end of the lead body. The flexibility is defined by the pattern and/or the geometry of the formed recesses.

According to yet another embodiment, the present invention is a medical electrical lead including at least one bonding region where a strong axial load bearing material capable of thin walled extrusion or molding is necessary to transmit an axial load through a lead body segment which does not allow for thick insulators. The bonding region includes a tubular member having at least one recess formed in an outer surface. The tubular member is capable of increasing bonding strength through mechanical joining via the recesses formed therein.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a medical electrical lead according to an embodiment of the present invention.

FIG. 2 is a cross-sectional view taken along the length of a lead body according to an embodiment of the present invention.

FIG. 3 is an end, cross-sectional view of a lead body according to an embodiment of the present invention.

FIG. 4 is a schematic view of a reinforcing member according to an embodiment of the present invention.

FIG. 5 is a partial cut-away view of a portion of a lead body according to yet another embodiment of the present invention.

FIGS. 6A-6F are schematic views of a portion of a reinforcing member including a plurality of recesses according to various embodiments of the present invention.

FIGS. 7A-7F are schematic views of a portion of a reinforcing member including a plurality of recesses according to various embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense.

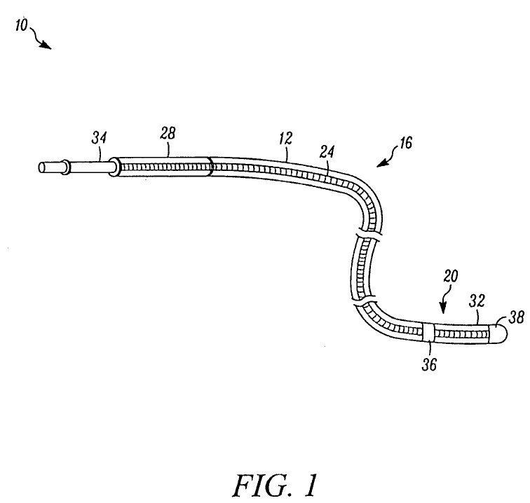

FIG. 1 is a partial cross-sectional view of a medical electrical lead 10, according to an embodiment of the present invention. Medical electrical lead 10 includes an elongated, flexible lead body 12 having a proximal portion 16 and a distal portion 20. In one embodiment of the present invention, the lead body 12 includes a lumen for receiving a guiding element such as a guidewire or a stylet.

Medical electrical lead 10 also includes one or more conductors 24, such as a coiled conductor, extending from a proximal end 28 to a distal end 32 of the lead body 12. The proximal end 28 is configured to be operatively connected to a pulse generator via a connector 34. Conductor 24 can be generally helical in configuration and can include one or more conductive wires or filaments. The conductor or conductors 24 can be coupled to one or more electrodes 36 and 38. The electrodes 36 and 38 can be coated with or formed from platinum, stainless steel MP35N, a platinum-iridium alloy, titanium or another similar conductive material. According to one embodiment, the outer diameter of the lead body ranges from about 2 to about 6 French. According to a further embodiment of the present invention, the outer diameter of the lead body is less than about 5 French.

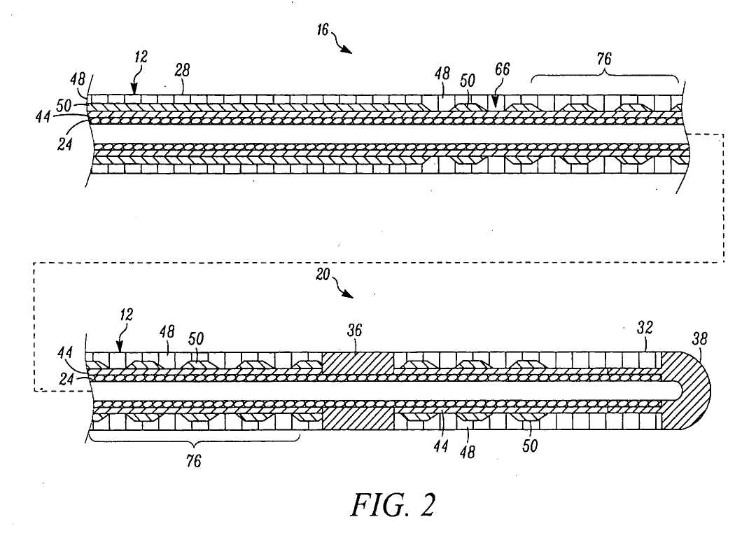

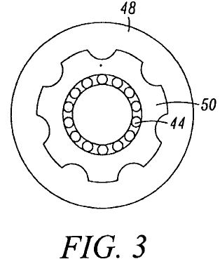

FIG. 2 is a detailed, cross-sectional view taken along a longitudinal axis of a lead body 12 according to an embodiment of the present invention. FIG. 3 is an end cross-sectional view of a lead body 12 according to an embodiment of the present invention. The lead body 12 can be fabricated according to those methods and techniques known to those of skill in the art.

According to one embodiment, as shown in FIGS. 2 and 3, the lead body 12 includes a conductor 24, a first insulator 44, an outer insulator 48, and a reinforcing member 50. The first insulator 44 is substantially disposed over the conductor 24 such that it extends from a proximal end 28 to a distal end 32 of the lead body 12. The first insulator 44 can be fabricated from a wide variety of insulative materials, examples of which include: silicone, SIBS, polyurethane, PTFE, ETFE, and other similar flexible insulating polymers. According to yet another embodiment, the first insulator 44 may not be required if the conductor need not be insulated or if the conductor does not have any incompatibilities with the reinforcing member 50.

According to one embodiment of the present invention, the reinforcing member 50 can be disposed over at least a portion of the first insulator 44 at a location between the first insulator 44 and the outer insulation 48 of the lead body 12. According to a further embodiment of the present invention, the reinforcing member 50 can be disposed immediately adjacent to the first insulator 44. According to yet another embodiment, one or more additional insulators may separate the reinforcing member 50 from the first insulator.

The outer insulator 48 is disposed over at least a portion of the reinforcing member 50 and extends substantially from the proximal end 28 to the distal end 32 of the lead body 12. The outer insulator 48 can be silicone, polyurethane, PTFE, SIBS or a combination thereof. According to one embodiment of the present invention, the outer insulator may be disposed immediately adjacent to the reinforcing member 48. According to other embodiments, the outer insulator may be separated from the reinforcing member 50 by one or more additional insulator layers.

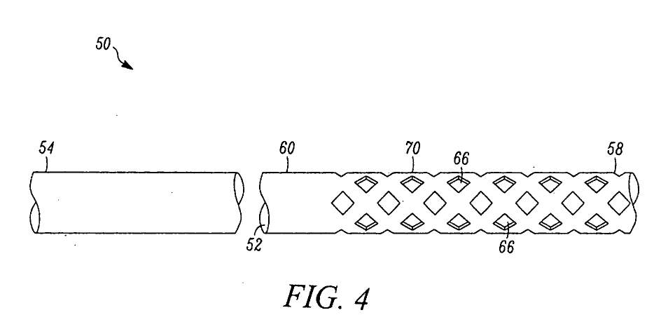

FIG. 4 is a schematic view of the reinforcing member 50 according to an embodiment of the present invention. As shown in FIG. 4, the reinforcing member 50 includes a tubular member 60 defining a lumen 52 extending between a proximal end 54 and a distal end 58. The tubular member 60 includes at least one recess 66 formed in an outer surface 70 of the tubular member 60.

The recesses 66 result from either mechanical, chemical, or laser removal of material from the outer surface 70 of the tubular member 60. Thus, thinning of the tubular wall from a first region to a second region can also be considered a recess according to embodiments of the present invention. According to one embodiment of the present invention, the recesses 66 are depressions formed in the outer surface 70 of the tubular member 60. According to another embodiment, the recesses 66 are orifices that extend through the outer surface 70 of the tubular member 60 and into the lumen 52. The pattern, density, area, and location of the recesses 66 formed in the outer surface 70 can affect the flexibility of the reinforcing member 50, thus affecting the overall flexibility of a lead 10 when used in constructing the lead body 12. The pattern, density, area and location of the recesses 66 can be selected depending on the desired flexibility and other characteristics of the lead body 12. According to one embodiment, the flexibility of the reinforcing member 50 increases in direct proportion to the number of recesses 66 formed in the outer surface 70 of the tubular member 60.

The tubular member 60 can be made from a mechanically stiff polymeric material including polymers and polymer composites. The tubular member 60 should be fabricated from a material capable of withstanding the axial load requirements of the lead body even with one or more recesses 66 formed in an outer surface 70 of the tubular member 60. According to one embodiment, the tubular member 60 should be capable of withstanding axial loads greater than about 1 lb with a permanent deformation ranging from about 5% to about 10%. According to another embodiment of the present invention, the tubular member 50 should be capable of withstanding axial loads greater than about 2 lbs with a permanent deformation ranging from about 5 to about 10%. Exemplary materials include polycarbonates, polyacrylates, polyurethanes, polyesters, polyamides, polyethylenes, polypropylenes, polyvinylchloride, and polytetrafluoroethylene. According to one exemplary embodiment, the polymeric material used to fabricate the tubular member 60 is a polyurethane. According to yet further embodiments of the present invention, Pellethane 2363 55D or Elasthane 55D may be used to construct the tubular member 60. According to yet another further embodiment of the present invention, polytetrafluoroethylene (PTFE) may be used to construct the tubular member.

Additionally, the wall thickness of the tubular member 60 should be sufficiently thin while at the same time maintaining the tensile strength and/or the tear resistance of the tubular member 60. According to one embodiment the wall thickness of the tubular member 60 ranges from about 0.001 to about 0.010 inches.

According to one embodiment of the present invention, as shown in FIGS. 2 and 4, the reinforcing member 50 extends from substantially the proximal end 28 to the distal end 32 of the lead body 12. The outer surface 70 of the tubular member 60 located at or near a proximal portion 16 of the lead body 12 is free from any recesses formed therein and is smooth. Moving in a distal direction from the proximal end 28 to the distal end 32 of the lead body 12, recesses are formed in the sheath, increasing its flexibility. Flexibility of the reinforcing member 50 can be increased from a proximal end 28 to a distal end 32 of the lead body 12 resulting in the distal portion 20 of the lead body 16 having a greater flexibility than the proximal portion 16 of the lead body 12. According to one embodiment of the present invention, at the distal portion 20 and/or distal end 32 of the lead body 12, the flexibility of the reinforcing member 50 can be sufficiently controlled such that it adopts the stiffness of a stylet of other guiding member inserted therein.

According to another embodiment of the present invention, as best shown in FIG. 2, the recesses 66 in the outer surface 70 of the reinforcing member 50 define one or more discrete flexibility regions 76 along the different portions of the lead body 12. The configuration, pattern, density, area and location of the recesses 66 can be used to determine the flexibility of the flexibility region(s) 76.

According to yet another embodiment of the present invention, as best viewed in FIG. 2, a flexibility region 76 as defined by the recesses 66 in the outer surface 70 of the reinforcing member 50 may be disposed about one or more electrodes 36 or 38 located on the lead body 12 in order to maintain flexibility of the lead body in the electrode region.

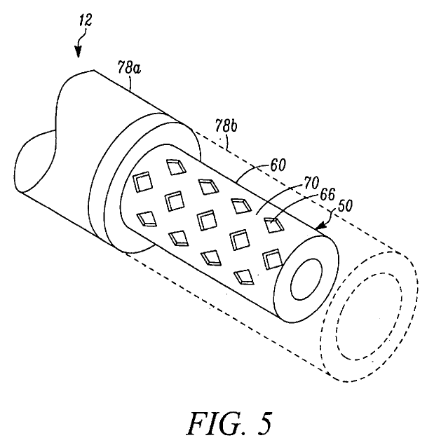

FIG. 5 is a partial cut-away view of a portion of a lead body 12 including the reinforcing member 50, according to an embodiment of the present invention. The reinforcing member 50 includes tubular member 60 having a plurality of recesses 66 formed in an outer surface 70. According one embodiment of the present invention, as shown in FIG. 5, tubular member 60 provides a bonding substrate for bonding a layer of a first material 78a adjacent to a layer of second material 78b, shown in phantom. The recesses 66 formed in the outer layer 70 of the tubular member 60 can create a mechanical interconnect via an adhesive material between the tubular member 60 and the adjoining insulative or non-insulative layer 78a or 78b. The materials 78a and 78b can be non-insulative or insulative. According to one embodiment of the present invention, the first material 78a can be polyurethane and the second material 78b can be silicone.

According to another embodiment of the present invention, the recesses 66 formed in the outer surface 70 of the tubular member 60 may also provide a mechanism for strengthening the mechanical interlocking of the insulative or non-insulative layers of material to the lead body 12 by increasing the amount of surface area available for adhesion. Increasing the mechanical boding strength may also facilitate strengthening the transition region from the a first material 78a to a second material 78b. According to yet another embodiment of the present invention, the tubular member 60 including any recesses 66 formed in an outer surface 70 thereof may provide at least one bonding region where a strong axial load bearing material capable of thin walled extrusion or molding is necessary to transmit an axial load through a lead body segment which does not allow for thick insulators.

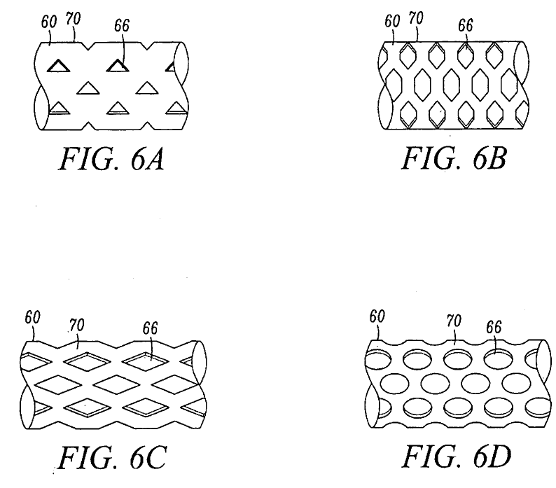

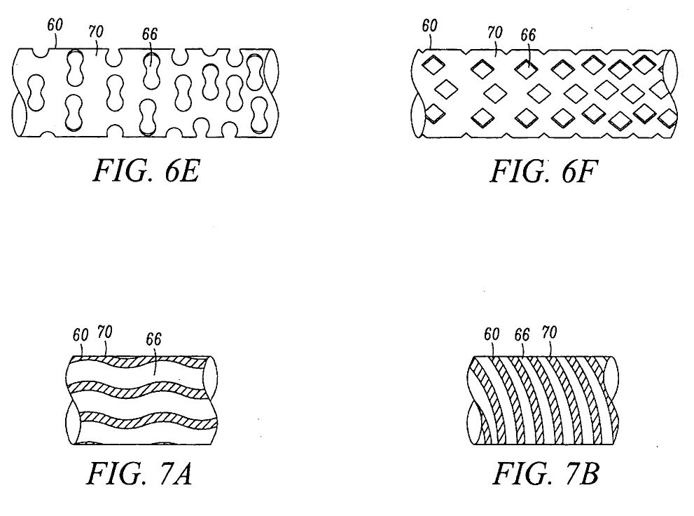

FIGS. 6A-6F are schematic views of portions of the tubular member 60 in which recesses 66 are formed in an outer surface 70 thereof according to various embodiments of the present invention. The recesses 66 whether they include depressions, orifices, or a combination thereof, can be formed in a variety configurations and patterns. The configuration, pattern, density, area and location of the recesses 66 can be used to control the desired flexibility and other desired characteristics of the lead body 12. According to one embodiment of the present invention, the recesses 66 form a non-random, ordered pattern on or in the outer surface 70 of the tubular member 60. According to another embodiment of the present invention, as shown in FIGS. 4 and 6A-6F, the recesses 66 have a polygonal configuration. The polygonal configuration may include a number of polygonal shapes including, but not limited to, the following: trigonal, tetragonal, pentagonal, hexagonal, dog-bone or dog-legged shaped, and elongated variations thereof. As shown in FIG. 6F, the number of recesses 66 formed in the outer surface 70 of the tubular member 60 may increase in number when moving in a distal direction along the tubular member 60.

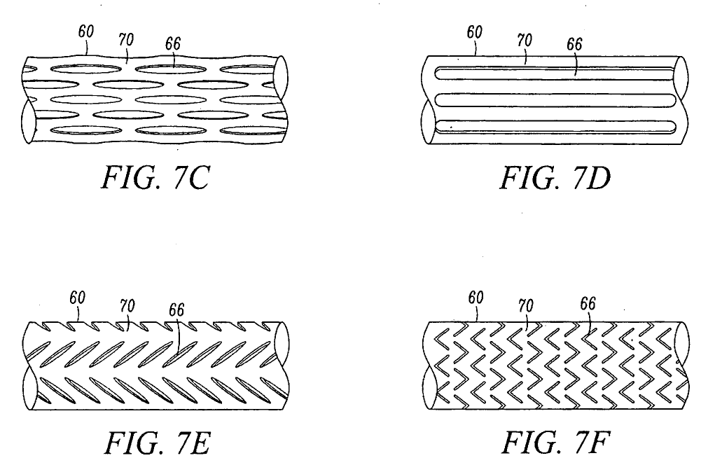

FIGS. 7A-7F are schematic views of portions of the tubular member 60 in which recesses 66 are formed in an outer surface 70 thereof according to yet other embodiments of the present invention. As shown in FIGS. 7A and 7B, the recesses 66 may have a sinusoidal or helical configuration longitudinally extending in a distal direction along at least a portion of the tubular member 60. According to other embodiments of the present invention, as shown in FIGS. 7C-7F, the recesses 66 may be a longitudinal slit formed in the outer surface 70 of the tubular member 70. The longitudinal slits can vary in number and length and generally extend in a distal direction along at least a portion of the tubular member 60. According to yet further embodiments of the present invention, as shown in FIGS. 7E and 7F, the longitudinal slits may be angled.

According to various embodiment of the present invention, the recesses 66 are formed by the mechanical or chemical removal of material from the outer surface 70 of the tubular member 60. According to another embodiment, the recesses 66 are formed in the outer surface 70 of the reinforcing member 50 using laser radiation. Laser radiation vaporizes the polymer material to be removed, while producing little or no mechanical or thermal effects on the remaining adjacent material. Because the material is vaporized by laser radiation, the cut material is not physically displaced or deformed as it is when mechanical cutting methods are used. Thermal effects are minimized or eliminated by the laser radiation, the polymeric material is not melted, and the beading up of melted polymeric material produced by thermal processing is avoided. Therefore, a recess produced using laser ablation reserves a smooth surface and the adjacent polymeric material is undisturbed so that the original outer diameter of the sheath is not increased. Additionally, laser removal of the polymeric material offers precise control over the removal of shaft material in order to vary the reinforcing member characteristics in a repeatable and controllable manner. Lasers used in the invention must be able to produce radiation capable of vaporizing polyurethane or similar materials. Selection of the wavelength and the appropriate optical set-up for imaging the laser beam on the reinforcing member shaft facilitates precise control over the removal of material in a selected area or pattern.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A medical electrical lead comprising;
a lead body including a proximal end configured to be connected to a pulse generator and a distal end;
a conductor extending from the proximal end to the distal end of the lead body;
at least one electrode operatively connected to the conductor;
a reinforcing member disposed over at least a portion of the conductor, the reinforcing member comprising a tubular member defining a lumen and including an outer surface having at least one recess formed therein; and
an outer insulator disposed over at least a portion of the reinforcing member.

2. The medical electrical lead according to claim 1, further comprising a first insulator disposed over a portion of the conductor, wherein the reinforcing member is disposed over at least a portion of the first insulator.

3. The medical electrical lead according to claim 1, wherein the recesses extend through the outer surface into the lumen of the tubular member.

4. The medical electrical lead according to claim 1, wherein the recesses form a non-random, ordered pattern in the outer surface of the tubular member.

5. The medical electrical lead according to claim 1, wherein the tubular member comprises a polyurethane.

6. The medical electrical lead according to claim 1, wherein the tubular member is capable of withstanding axial loads of greater than about 1 lbs with a permanent deformation ranging from about 5% to about 10%.

7. The medical electrical lead according to claim 1, wherein the tubular member is capable of withstanding axial loads of greater than about 2 lbs with a permanent deformation ranging from about 5% to about 10%.

8. The medical electrical lead according to claim 1, wherein the recesses formed in the outer surface of the tubular member define one or more discrete flexibility regions located along the lead body.

9. The medical electrical lead according to claim 8 wherein at least one flexibility region is located adjacent to the electrode.

10. The medical electrical lead according to claim 1, wherein a number of recesses formed in the outer surface of the tubular member increases from the proximal end to the distal end of the lead body.

11. The medical electrical lead according to claim 1, wherein the recesses comprise a polygonal configuration.

12. The medical electrical lead according to claim 1, wherein the recesses longitudinally extend in a distal direction along at least a portion of the outer surface of the tubular member.

13. The medical electrical lead according to claim 1, wherein the recesses comprise a substantially helical configuration longitudinally extending in a distal direction along at least a portion of the outer surface of the tubular member.

14. The medical electrical lead according to claim 1, wherein flexibility of the lead body increases in direct proportion to an amount of material removed to form the recesses in the outer surface of the tubular member.

15. The medical electrical lead according to claim 1, wherein a wall thickness of the tubular member ranges from about 0.001 to about 0.010 inches.

16. The medical electrical lead according to claim 1, wherein the reinforcing member extends from substantially the proximal end to the distal end of the lead body.

17. The medical electrical lead according to claim 1, wherein an outer diameter of the lead body ranges from about 2 to about 6 French.

18. The medical electrical lead according to claim 1, wherein an outer diameter of the lead body is less than about 5 French.

19. A medical electrical lead comprising:
a lead body including a proximal end configured to be connected to a pulse generator and a distal end;
a conductor extending from the proximal end to the distal end of the lead body;
a first insulator disposed over the conductor;
at least one electrode operatively connected to the conductor; and
a reinforcing member disposed over the first insulator and extending from substantially the proximal end to the distal end of the lead body, the reinforcing member comprising a tubular member defining a lumen and including an outer surface having a non-random, ordered pattern of a plurality of recesses extending through the outer surface into the lumen; and
an outer insulator disposed over at least a portion of the reinforcing member.

20. The medical electrical lead according to claim 19, wherein the recesses formed in the outer surface of the tubular member define one or more discrete flexibility regions located along the lead body.

21. The medical electrical lead according to claim 19, wherein a number of recesses formed in the outer surface of the tubular member increase from the proximal end to the distal end of the lead body.

22. The medical electrical lead according to claim 19, wherein an outer diameter of the lead body is less than about 5 French, and the lead body is capable of with standing axial loads of greater than about 1 lbs with a permanent deformation ranging from about 5% to about 10%.

23. A medical electrical lead comprising:
a lead body including a proximal end configured to be connected to a pulse generator and a distal end;
a conductor extending from the proximal end to the distal end of the lead body;
at least one electrode operatively connected to the conductor; and
at least one flexibility region located between the proximal end and the distal end of the lead body.

24. The medical electrical lead according to claim 23, wherein the flexibility region comprises at least one recess formed in an outer surface of a tubular member disposed over at least a portion of the conductor.

25. A medical electrical lead comprising a lead body including a proximal end configured to be connected to a pulse generator and a distal end;
a lead body including a proximal end configured to be connected to a pulse generator and a distal end;
a conductor extending from the proximal end to the distal end of the lead body;
at least one electrode operatively connected to the conductor;
a bonding substrate having variable flexibility disposed over the conductor, the substrate providing an increased bonding surface area for adhesion of an insulator; and
an insulative material bonded to the bonding substrate.
